(19) United States
(12) Patent Application Publication
(10) Pub. No.: US 2012/0109021 A1
(43) Pub. Date: May 3, 2012

(54) RENAL DENERVATION CATHETER EMPLOYING ACOUSTIC WAVE GENERATOR ARRANGEMENT

(76) Inventors: Roger Hastings, Maple Grove, MN (US); Mark L. Jenson, Greenfield, MN (US)

(21) Appl. No.: 13/283,203

(22) Filed: Oct. 27, 2011

Related U.S. Application Data
(60) Provisional application No. 61/407,320, filed on Oct. 27, 2010.

(51) Int. Cl. A61N 7/00

(52) U.S. Cl. 601/2

(57) ABSTRACT

A transducer supported by a positioning arrangement is placed within a renal artery at a desired location that is a predetermined distance from a reflector equal to an odd number of quarter wavelengths of acoustic energy emitted by the transducer. The positioning arrangement is actuated to transition from a low-profile introduction configuration to a deployed configuration within the renal artery thereby stabilizing the transducer at a desired location. Acoustic energy is emitted by the transducer so that it propagates axially along an outer surface of the target vessel to impinge the reflector, which can be biological or artificial. The emitted energy builds up to resonance at a point of reflection defined by a location of the reflector, and the amount of energy build up is sufficient to ablate perivascular renal nerves in the vicinity of the reflector.

### RELATED PATENT DOCUMENTS

This application claims the benefit of Provisional Patent Application Ser. No. 61/407,320 filed Oct. 27, 2010, to which priority is claimed pursuant to 35 U.S.C. §119(e) and which is hereby incorporated herein by reference.

### SUMMARY

Embodiments of the disclosure are directed to an apparatus which includes a flexible shaft having a proximal end, a distal end, a length, and a lumen arrangement extending between the proximal and distal ends. The length of the shaft is sufficient to access target tissue of the body relative to a percutaneous access location. The target tissue is capable of supporting standing waves. A positioning structure is provided at a distal end of the shaft. A transducer is supported by the positioning structure and arranged to emit acoustic energy so that it impinges a reflector within or proximate the target tissue. The acoustic energy emitted by the transducer produces standing waves in the target tissue and one or more loops of high amplitude acoustic energy sufficient to ablate the target tissue.

According to various embodiments, an apparatus includes a flexible shaft having a proximal end, a distal end, a length, and a lumen arrangement extending between the proximal and distal ends. The length of the shaft is sufficient to access a renal artery relative to a percutaneous access location of the body. A positioning structure is provided at a distal end of the shaft and is transformable between a low-profile introduction configuration and a deployed configuration. A transducer is supported by the positioning structure and arranged to emit acoustic energy so that it propagates axially along an outer surface of the renal artery to impinge a reflector. The acoustic energy emitted by the transducer produces standing waves on perivascular renal nerves and one or more loops of high amplitude acoustic energy sufficient to ablate the perivascular renal nerves.

Other embodiments are directed to a method involving positioning a transducer within or proximate target tissue that supports standing waves at a location relative to a reflector. The method also involves emitting acoustic energy by the transducer so that it impinges the reflector, and ablating the target tissue by producing standing waves in the target tissue and one or more loops of high amplitude acoustic energy sufficient to ablate the target tissue. The method may involve adjusting a frequency of the emitted acoustic energy to achieve resonance of the target tissue. In some method embodiments, the transducer is positioned within a renal artery, the acoustic energy is emitted so that it propagates axially along an outer surface of the renal artery to impinge the reflector, and perivascular renal nerves are ablated by producing standing waves on the renal nerves and one or more loops of high amplitude acoustic energy sufficient to ablate the renal nerves.

In accordance with various embodiments, an apparatus includes a catheter having a flexible shaft with a proximal end, a distal end, a length, and a lumen arrangement extending between the proximal and distal ends. The length of the shaft is sufficient to access a renal artery relative to a percutaneous access location of the body. A cylindrical ultrasound transducer is provided at a distal end of the shaft and dimensioned for placement within a lumen of the renal artery. A positioning structure is provided at a distal end of the shaft and transformable between a low-profile introduction configuration and a deployed configuration. The positioning structure is configured to center the transducer in the lumen of the renal artery when in the deployed configuration. The transducer is configured to generate bursts of ultrasound energy and repeatedly emit the ultrasound energy bursts at a resonance frequency of the renal nerves to generate standing waves on the renal nerves of sufficient amplitude to mechanically ablate the renal nerves.

In further embodiments, a method involves positioning a cylindrical ultrasound transducer in a lumen of a renal artery at a central location of the lumen. The method also involves generating bursts of ultrasound energy, and repeatedly emitting the ultrasound energy bursts at a resonance frequency of the renal nerves to generate standing waves on the renal nerves of sufficient amplitude to mechanically ablate the renal nerves.

Some embodiments of the disclosure are directed to an apparatus which includes a catheter comprising a flexible shaft having a proximal end, a distal end, a length, and a lumen arrangement extending between the proximal and distal ends. The length of the shaft is sufficient to access a target vessel of the body relative to a percutaneous access location of the body. A transducer arrangement is provided at a distal end of the shaft and includes a positioning structure and a transducer. The positioning structure is transformable between a low-profile introduction configuration and a deployed configuration. The transducer is supported by the positioning structure and configured to emit acoustic energy having a wavelength and to direct the emitted acoustic energy so that it propagates axially along an outer surface of the target vessel to impinge a reflector situated a predetermined distance from the transducer. The predetermined distance is equal to an odd number of quarter wavelengths of the energy emitted by the transducer. The acoustic energy emitted by the transducer builds up to resonance at a point of reflection defined by a location of the reflector, and this acoustic energy build up is sufficient to ablate target tissue in the vicinity of the reflector.

According to various embodiments, an apparatus includes a catheter comprising a flexible shaft having a proximal end, a distal end, a length, and a lumen arrangement extending between the proximal and distal ends. The length of the shaft is sufficient to access a renal artery relative to a percutaneous access location of the body. A transducer arrangement is provided at a distal end of the shaft and includes a positioning structure and a transducer. The positioning structure is transformable between a low-profile introduction configuration and a deployed configuration. The transducer is supported by the positioning structure and configured to emit acoustic energy having a wavelength and to direct the acoustic emitted energy so that it propagates axially along an outer surface of the renal artery to impinge a reflector situated a predetermined distance from the transducer. The predetermined distance is equal to an odd number of quarter wavelengths of the energy emitted by the transducer. The acoustic energy emitted by the transducer builds up to resonance at a point of reflection defined by a location of the reflector, and the amount of acoustic energy build up is sufficient to ablate perivascular renal nerve tissue in the vicinity of the reflector.

In accordance with other embodiments, a method involves positioning a transducer supported by a positioning arrangement within a target vessel at a desired location that is a predetermined distance equal to an odd number of quarter wavelengths of acoustic energy emitted by the transducer from a reflector. The method also involves actuating the positioning arrangement to transition from a low-profile introduction configuration to a deployed configuration within the target vessel thereby stabilizing the transducer at the desired location. The method further involves emitting acoustic energy by the transducer so that it propagates axially along an outer surface of the target vessel to impinge the reflector. The emitted acoustic energy builds up to resonance at a point of reflection defined by a location of the reflector, and the amount of acoustic energy build up is sufficient to ablate target tissue in the vicinity of the reflector. The target vessel may be a renal artery, and the target tissue may include perivascular renal nerve tissue.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 illustrates a distal end of an ablation catheter which includes an electrode and an integral contrast dye injection arrangement in accordance with various embodiments;

FIGS. 5 and 6 are simplified illustrations depicting the positional relationship between an acoustic transducer and a reflector for producing single-mode and multiple-mode resonant acoustic energy sufficient to ablate target tissue of the body in accordance with various embodiments;

FIG. 7 illustrates a vibratory renal denervation catheter that employs a balloon arrangement to support an acoustic transducer and to form a reflection feature at a specified distance from the transducer and within a renal artery in accordance with various embodiments;

FIG. 8 illustrates an acoustic transducer of a vibratory renal denervation catheter positioned at an ostium of a renal artery, the acoustic transducer using a kidney and/or main bifurcation as an acoustic reflector in accordance with various embodiments;

FIG. 9 shows details of the transducer of FIG. 8 in accordance with various embodiments;

FIG. 10 illustrates an electromagnetic acoustic generator in accordance with various embodiments;

FIGS. 11A-11C illustrate an embodiment of a transducer assembly supported by a mesh structure in three different configurations in accordance with various embodiments;

FIG. 12 shows the transducer illustrated in FIGS. 11A-11B in its deployed configuration in accordance with various embodiments;

FIG. 13 illustrates a cylindrical ultrasound transducer and a positioning arrangement provided at a distal end of a flexible shaft of an ablation catheter and positioned within a renal artery in accordance with various embodiments;

FIG. 14 is a graph of acoustic power versus time for acoustic pulses generated by a cylindrical ultrasound transducer excited at its resonant frequency, the acoustic pulses being repeated at a resonance frequency of a renal nerve to mechanically disrupt the renal nerve in accordance with various embodiments; and

FIG. 15 illustrates a system for ablating tissues of the body, such as renal nerve tissue, using vibratory action resulting from acoustic energy excitation of the target tissue in accordance with various embodiments.

### DETAILED DESCRIPTION

Embodiments of the disclosure are directed to apparatuses and methods for ablating target tissue of the body using the acoustic energy that does not cause heating or damage to surrounding tissues. Embodiments of the disclosure are directed to apparatuses and methods for ablating perivascular renal nerves using a disruptive vibratory mechanism that mechanically ablates the renal nerves, such as for the treatment of hypertension. Apparatuses and methods described herein are directed to the use of resonant acoustic energy for ablating tissues of the body, such as renal nerves, without heating or damaging surrounding tissues.

According to various embodiments, an ablation catheter supports an acoustic transducer at its distal end which is configured to generate acoustic energy in the kilohertz range. The ablation catheter is advanced into the body so that the acoustic transducer is positioned within or proximate target tissue to be ablated. The target tissue is capable of supporting standing waves. The acoustic transducer is positioned to emit acoustic energy so that it impinges a reflector within or proximate the target tissue. The acoustic energy emitted by the transducer produces standing waves in the target tissue and one or more loops of high amplitude acoustic energy sufficient to mechanically ablate the target tissue. In some embodiments, the acoustic transducer is advanced through the vasculature and positioned at an ostium of the renal artery. The acoustic transducer is positioned so that the emitted acoustic energy propagates axially along an outer surface of the renal artery to impinge the reflector, producing standing waves on perivascular renal nerves and one or more loops of high amplitude acoustic energy sufficient to mechanically ablate the perivascular renal nerves.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

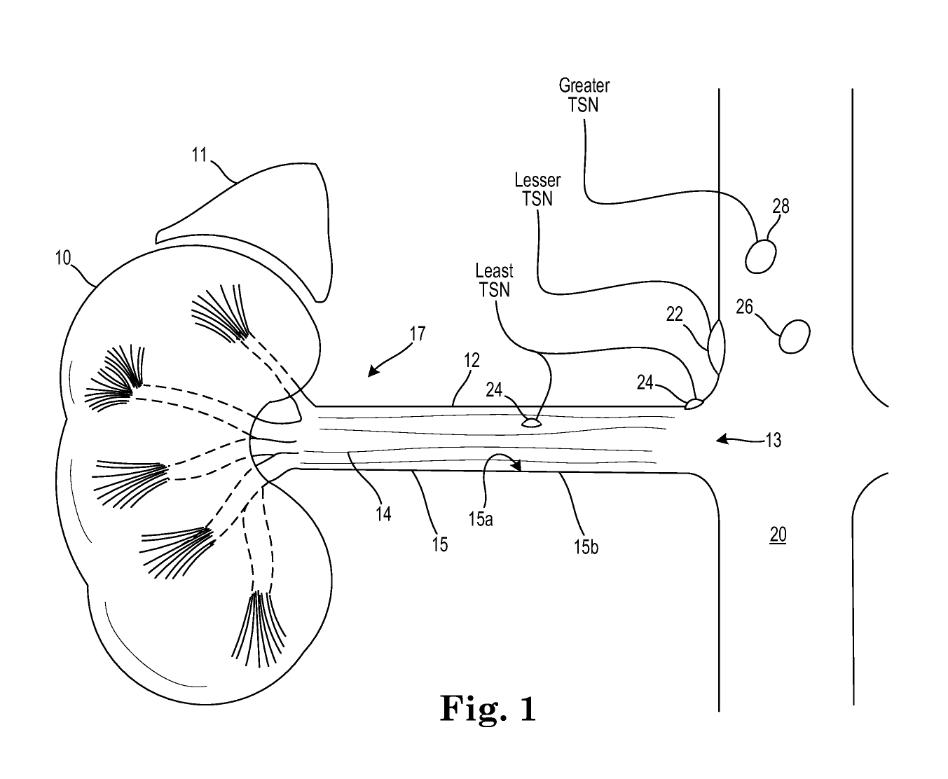

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

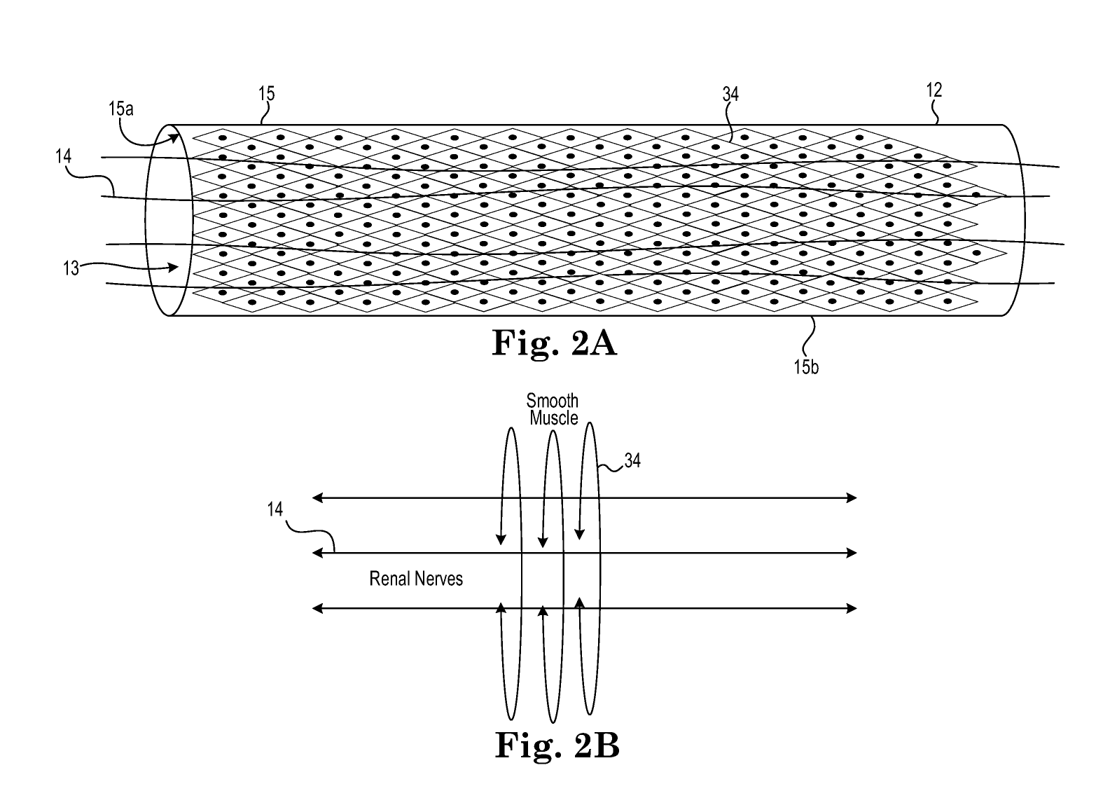

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

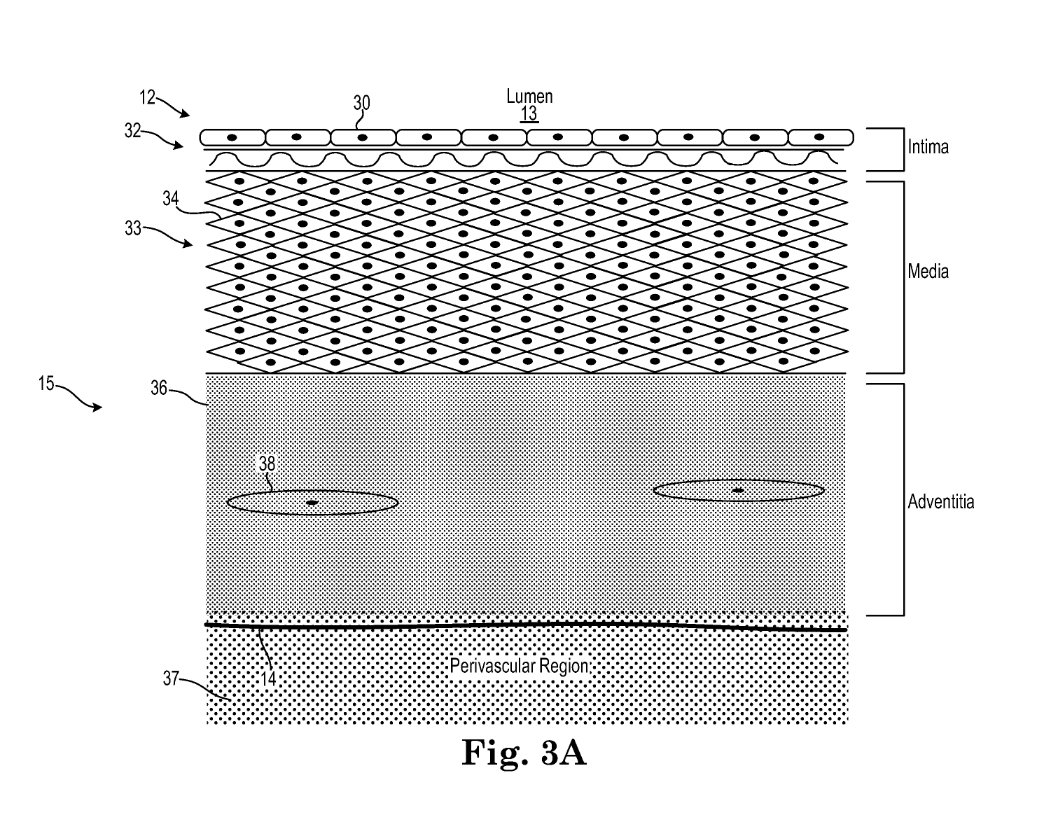

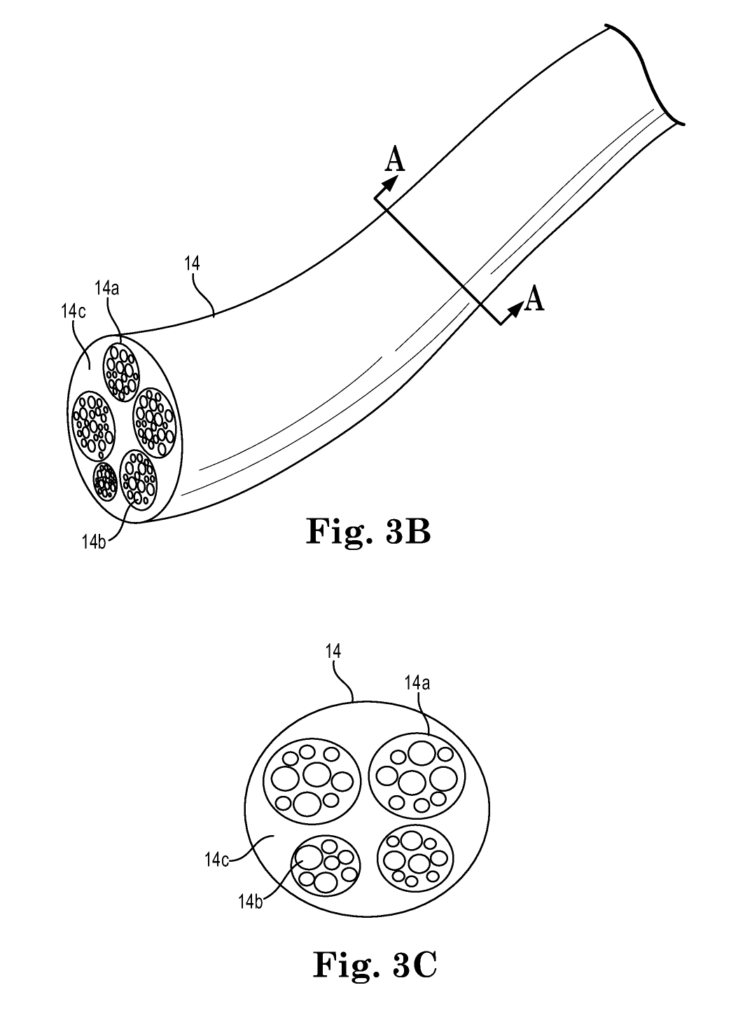

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b is preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

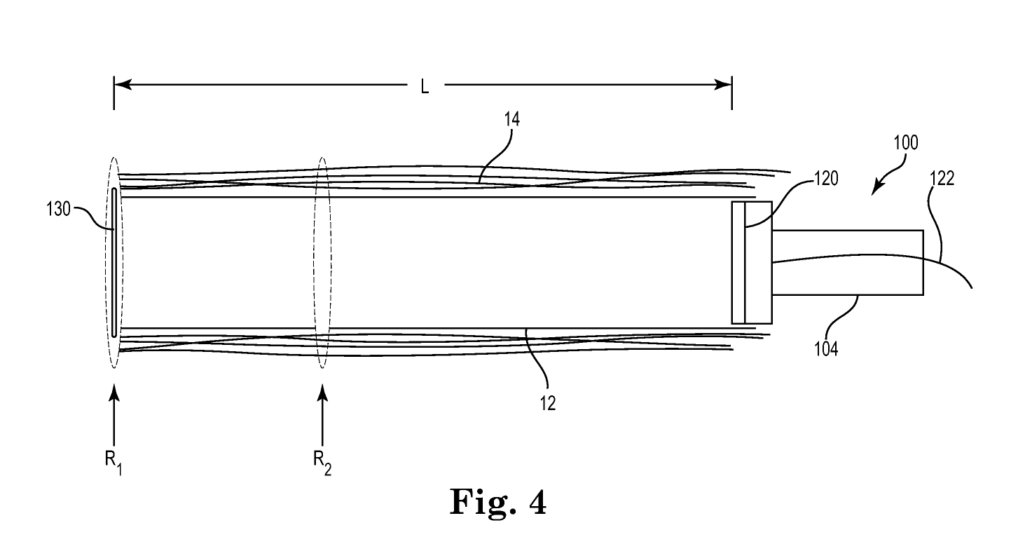

With reference to FIG. 4, a transducer 120 of an ablation catheter 100 is shown deployed within a vessel 12 of the body. The catheter 100 includes a flexible shaft 104 having a length sufficient to access a target vessel 12 relative to a percutaneous access location of the body. The transducer 120 is electrically coupled to a conductor 122 provided in a lumen arrangement of the shaft 104. A positioning structure (not shown for simplicity in FIG. 4) is preferably incorporated at the distal end of the shaft 104 and configured to support the transducer 120 when deployed within the target vessel 12.

Referring for the moment to FIG. 3A, the media 32 of an artery, such as a renal artery 12, has a sound propagation speed and acoustic impedance that is greater than that of the adventitia 36 and of the fat surrounding the renal artery 12. These differences result in the wall of the artery 12 acting like a waveguide that keeps the acoustic energy outside the artery wall, which concentrates the acoustic energy in the perivascular space that includes renal nerves 14.

The transducer 120 is preferably configured to emit acoustic energy having a specified wavelength. The transducer 120 is configured to direct the emitted acoustic energy so that it propagates axially along an outer surface of the target vessel 12 to impinge a reflector 130 situated at a predetermined distance, L, from the transducer 120. The reflector 130 may be an artificial reflector, such as a component of an intravascular catheter apparatus, or a biological reflector, such as an organ of the body. The predetermined distance, L, is preferably equal to an odd number of quarter wavelengths of the acoustic energy emitted by the transducer 120.

The acoustic energy emitted by the transducer 120 builds up to resonance at a point of reflection, R₁, defined by a location of the reflector 130. The amount of acoustic energy buildup is sufficient to cause vibratory disruption of renal nerve fibers 14 in the vicinity of the reflector 130, preferably to a degree sufficient to physically separate the endoneurium tube of the renal nerve fibers 14. At resonance, vibratory damage to the renal nerves 14 can be induced at acoustic power levels that do not cause heating or damage to surrounding tissues.

Additional points of reflection can be created by positioning the transducer 120 at a predetermined distance, L, equal to n quarter wavelengths of the energy emitted by the transducer 120, where n is an odd number greater than one. For example, a second point of reflection, R₂, can be created by positioning the transducer 120 at a predetermined distance, L, equal to three-fourths of a wavelength of the energy emitted by the transducer 120.

It is understood that varying degrees of renal nerve disruption can be achieved by increasing or decreasing the acoustic power build up at the point or points of rejections (antinodes). For example, renal nerve ablation may be considered efficacious where the endoneurium is disrupted, but with the epineurium and the perineurium remaining intact. As another example, renal nerve ablation may be considered efficacious where all neural and supporting elements are disrupted, but the epineurium remains intact. In yet another example, renal nerve ablation may be considered efficacious where both the renal nerve fiber and the nerve sheath are disrupted.

The transducer 120 is preferably a transducer configured to emit acoustic energy, such as relatively low frequency acoustic energy in the kilohertz range. In some embodiments, the transducer 120 is configured to emit acoustic energy within a frequency range beginning within the human audio spectrum (e.g., ≧˜8 kHz) and extending into the low end of the ultrasonic spectrum (e.g., ≦150 kHz). In some embodiments, the frequency of the emitted acoustic energy can be fixed, while in other embodiments the frequency can be varied.

According to various embodiments, the transducer 120 includes an acoustic or vibration generator, such as an electromagnetic vibrator. In some embodiments, the acoustic or vibration generator is capable of producing acoustic energy of varying magnitude and across a range of frequencies, such as between about 5 kHz and about 200 kHz for example. The range of frequencies preferably includes frequencies that are known or expected to create standing waves on renal nerves or other target tissue of the body that supports standing waves. Such frequencies are typically determined by deduction or experimental measurements made in the laboratory, with the expectation that one or many resonances will be created in the renal nerves or target tissue during a frequency sweep.

In cases where a renal nerve 12 is constrained along its length, the point of constraint becomes a node. For example, a renal nerve 12 may be constrained as it enters the kidney. In such cases, an integral number of half wavelengths between the transducer 120 and the point of constraint is needed, which can be achieved by searching for a resonant frequency, even though the exact relationship of the wavelength to the length of the nerve 12 may not be known.

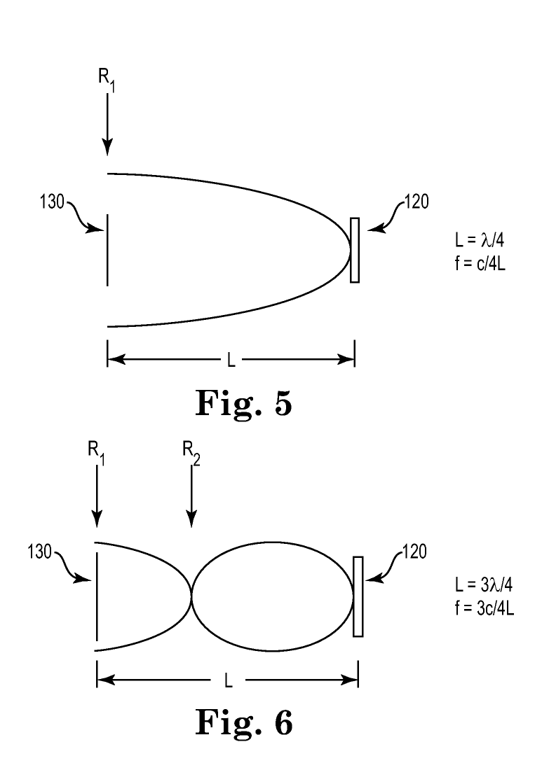

FIGS. 5 and 6 are simplified illustrations depicting the positional relationship between a transducer 120 and a reflector 130 for producing single-mode and multiple-mode resonant acoustic energy sufficient to ablate target tissue of the body in accordance with various embodiments. FIG. 5 illustrates the positional relationship between a transducer 120 and a reflector 130 needed to produce single-mode resonant acoustic energy. In FIG. 5, the transducer 120 represents a node of the compression wave amplitude. Loops, or intensity maxima, occur at distances equal to an odd number of quarter wavelengths from the transducer 120 (i.e., locations of antinodes). If a reflector 130 is placed at a loop, the amplitude of acoustic energy at the loop will build up to its resonance value.

In FIG. 5, the transducer 120 is positioned at a distance, L, relative to the reflector 130 equal to one quarter of a wavelength, λ. The relationship between the acoustic energy frequency, f, and speed of sound, c, needed for resonance in the scenario is given by f=c/4L. In this case, a single loop or intensity maxima, R₁, is created at a location corresponding to that of the reflector 130. In FIG. 6, the transducer 120 is positioned at a distance, L, relative to the reflector 130 equal to three quarters of a wavelength, λ. The relationship between the acoustic energy frequency and speed of sound in this scenario needed for resonance is given by f=3c/4L. In this case, two loops or intensity maxima are created; R₁ corresponding to a location of the reflector 130 and R₂, corresponding to a location equal to one-third of the distance, L, between the reflector 130 and the transducer 120. Any number of loops can be created when an odd number of quarter wavelengths fit within the length L. Ideally, the acoustic energy beam emitted by the transducer 120 would be reflected 180 degrees back from the reflector 130 to the transducer 120. In practice, a component of the emitted beam will be returned.

One or more sites along the renal artery 12 can thereby experience high amplitude acoustic energy sufficient in intensity to mechanically ablate perivascular renal nerve tissue 14 adjacent the renal artery 12. Performing renal denervation in accordance with various embodiments of the disclosure advantageously provides for acoustic energy delivery into the perivascular space from the outside of the renal artery 12 where the renal nerves 14 reside. Circumferential ablation can be achieved which insures complete renal nerve ablation. Use of resonant acoustic energy typically requires relatively small power input in comparison to conventional approaches, thus reducing the size and power requirements of the ablation device electronics. Such reduction in size and power requirements of the electronics enables implementation of self-powered renal ablation catheters that can be powered using conventional batteries. The vibratory ablation approaches disclosed herein may be incorporated in the self-powered renal ablation catheters disclosed in commonly owned co-pending U.S. patent application Ser. No. 13/227,446 filed on Sep. 7, 2011, which is incorporated herein by reference.

As described above, resonance amplitude of acoustic energy is created when an odd number of quarter wavelengths of the energy transmitted by the transducer 120 equals the separation, L, of the transducer 120 and the point of reflection, Rₙ. Given a speed of sound in tissue of about 1,500 msec, the frequencies needed for the first two modes (e.g., R₁ and R₂ in FIG. 4) when the separation, L, is 1 cm are 37.5 kHz and 112.5 kHz, respectively. When the separation, L, between the transducer 120 and reflector 130 is 2 cm, these resonant frequencies are 18.75 kHz and 56.25 kHz, respectively. Attenuation in tissue should be small at these relatively low frequencies, enabling high Q and resonance.

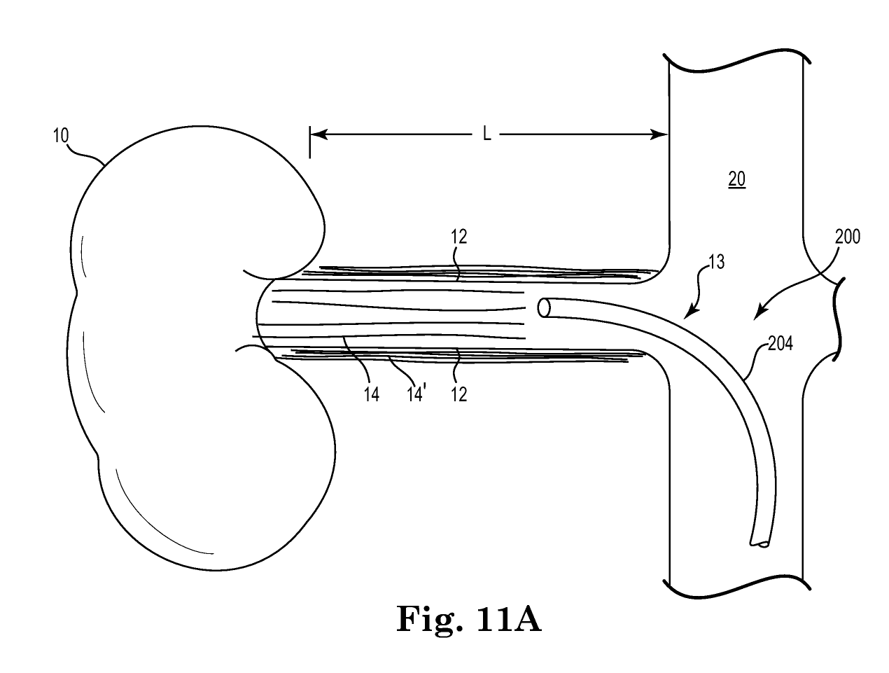

In accordance with various embodiments, the transducer 120 preferably includes an electromagnetic vibrator, which is particularly useful when the frequency of the emitted energy is relatively low (e.g., ≦20 kHz). An embodiment of an electromagnetic vibrator is described below with reference to FIG. 10. The transducer 120 is preferably implemented as a variable frequency transducer, which allows for adjustment of the wavelength to achieve a resonance frequency for a given separation distance, L, between the transducer 120 and reflector 130 (e.g., organ of the body or artificial component of an intravascular or implantable device).

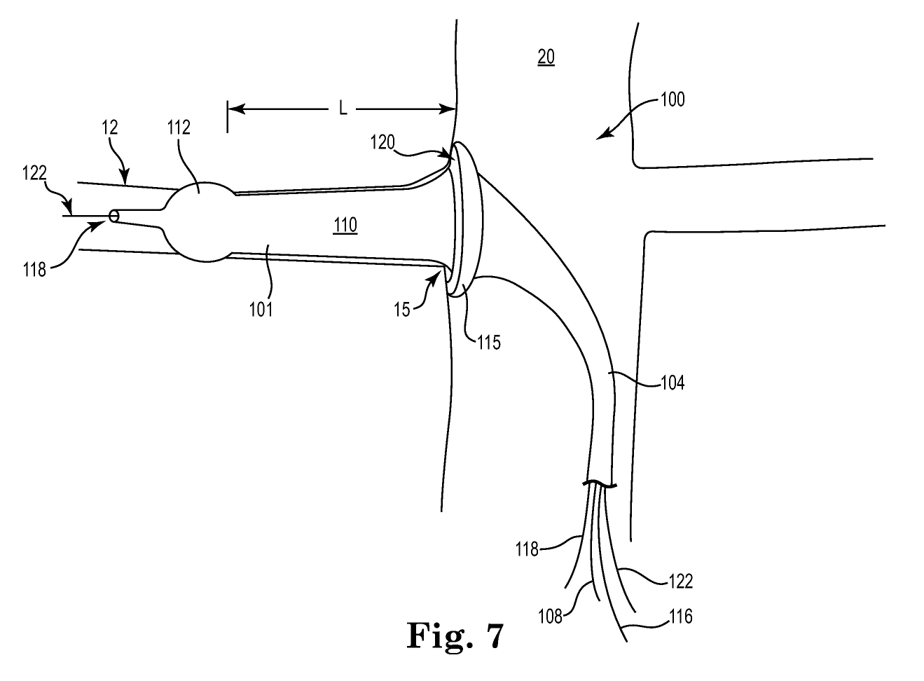

Turning now to FIG. 7, there is illustrated an ablation catheter 100 deployed in a renal artery 12 patient. The ablation catheter 100 includes a flexible shaft 104 having a length sufficient access the patient's renal artery 12 relative to a percutaneous access location. The ablation catheter 100 includes a balloon arrangement 101 provided a distal end of the shaft 104. In the embodiment shown in FIG. 7, the shaft 104 and the balloon arrangement 101 incorporate a lumen arrangement which includes a guide lumen 118 dimensioned to receive a guidewire 122. The guidewire 122 is typically used to locate the patient's renal artery 12 and advance the distal end of the ablation catheter 100 into the lumen of the renal artery 12. In some embodiments, a guidewire lumen is excluded, and the distal end of the ablation catheter 100 is advanced into the renal artery 12 using one or both of a guiding catheter and delivery sheath, for example.

The balloon arrangement 101 includes a first balloon section 115 dimensioned for abutting engagement with a wall of the aorta 20 and an ostium 15 of the renal artery 12. The first balloon section 115 is configured to abut the ostium 15 of the renal artery 12 and press a transducer 120 up against the wall of the aorta 20 at the aortorenal junction. The first balloon section 115 is fluidly coupled to an inflation lumen 116 which extends from the first balloon section 115 to a proximal end of the ablation catheter 100. A conductor 122 is provided in the lumen arrangement of the flexible shaft 104 and extends between the transducer 120 and a proximal end of the ablation catheter 100. In some embodiments, the conductor 122 comprises an electrical conductor. In other embodiments, the conductor 122 comprises a wire, ribbon, or other elongated structure capable of transmitting excitation vibrations from the proximal end of the ablation catheter 102 the transducer 120.

The balloon arrangement 101 further includes a second balloon section 110 dimension for deployment within the lumen of the renal artery 12. An inflation lumen 108 is fluidly coupled to the second balloon section 110 and extends to the proximal end of the ablation catheter 100. The second balloon section 110 includes a bulge feature 112, which is situated at a predetermined distance, L, from the transducer 120 when the second balloon section 110 is inflated. The bulge feature 112 is dimensioned to expand to a radius greater than a radius of the renal artery 12 and cause formation of a bump in the renal artery wall when the second balloon section 110 is pressurized. The bulge feature 112 forming the renal artery bump serves as a reflector. It is noted that in some embodiments, the first and second balloon sections 115 and 110 can be fluidly coupled to a common inflation lumen rather than separate inflation lumens 116 and 108.

After the first and second balloon sections 115 and 110 are inflated, the bulge feature 112 creates a bump in the renal artery wall located a predetermined distance, L, from the transducer 120. This predetermined distance, L, is equal to an odd number of quarter wavelengths of the energy emitted by the transducer 120. The transducer 120 is preferably a variable frequency transducer, such that the frequency of the emitted acoustic energy can be tuned to achieve resonance. The process of tuning the transducer frequency is preferably conducted using low amplitude acoustic energy emission, which serves to prevent any damage to tissues subjected to the acoustic energy emission. A parameter such as reflected power at the transducer resonant frequency may be monitored to detect resonance. After tuning the transducer frequency to achieve resonance, the amplitude of acoustic energy emission from the transducer 120 is increased so that the amount of acoustic energy buildup at one or more reflection points is sufficient to ablate perivascular renal nerve tissue in the vicinity of the one or more reflection points.

According to some embodiments, at least the bulge feature 112 of the second balloon section 110 is configured to receive a cryogen via a lumen provided in the shaft 104. A cryogen can be delivered to the bulge feature 112 to cause the formation of ice at the wall of the bulge feature 112. Formation of an ice ball at the bulge feature 112 causes the bulge feature 112 to more efficiently reflect acoustic energy emitted by the transducer 120. The bulge feature 112 or the entire second balloon section 110 can be configured as a cryoballoon. In some implementations, the cryoballoon 110/112 can be configured to receive a liquid biocompatible cryogen, such as cold sterile saline or cold Ringer's solution, which causes formation of the ice ball at the bulge feature 112 and is expelled into the blood flowing through the renal artery 12. In other implementations, the cryoballoon 110/112 can be constructed to provide phase-change cryothermal cooling by incorporating one or more orifices or narrowings to induce a phase change in a liquid cryogen supplied to the cryoballoon 110/112 (e.g., Joule-Thomson cooling). Spent gas resulting from the phase change of the liquid cryogen can be exhausted through an outlet fluidly coupled to an exhaust lumen that extends to the proximal end of the catheter shaft 104.

It is noted that one or more temperature sensors may be provided at the bulge feature 112 of the second balloon section 110 for measuring temperature approximating that of the renal artery wall adjacent the bulge feature 112. Also, marker bands can be situated on one or multiple parts of the balloon arrangement 101, such as the first and second balloon sections 115 and 110, and the catheter's shaft 104 to enable visualization for advancing the shaft 104 through vasculature and positioning the balloon arrangement 101 in the renal artery 12.

The progress and efficacy of perivascular renal nerve ablation can be monitored using the transducer 120, a separate intravascular or transvascular device, or an external device. For example, the transducer 120 or separate transducer at the distal end of the shaft 104, can include an ultrasound crystal transducer, for example, which can be used to characterize tissue changes without an actual visual image display. The changes can be detected and a simple indicator light on the handle of the ablation catheter 100 or on an external control system can illuminate to indicate “successful ablation,” for example. External systems may also be used to assess an ablation procedure including an MRI (magnetic resonance imaging) system, for example. Other external or internal monitoring approaches include acoustic imaging or other imaging, temperature monitoring, electrical impedance measurements, and acoustic impedance monitoring, for example.

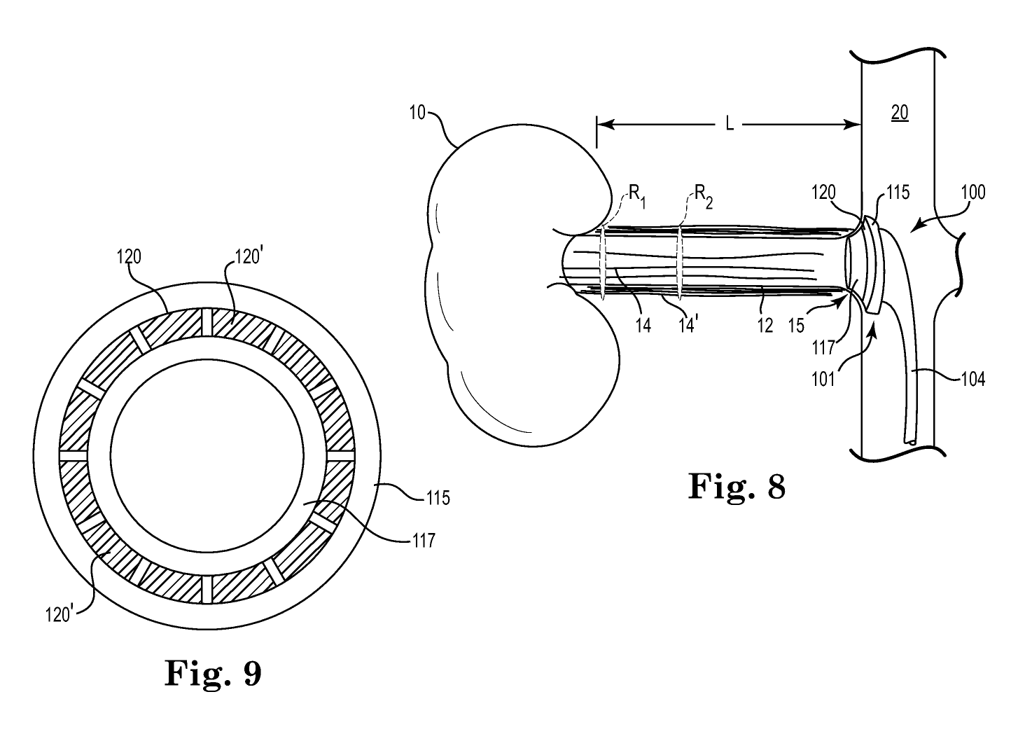

Turning now to FIG. 8, there is illustrated and ablation catheter 100 configured to ablate target tissue of the body using non-thermal acoustic energy in accordance with various embodiments. In the embodiment shown in FIG. 8, an ablation catheter 100 is shown deployed at the ostium 15 of a patient's renal artery 12. The ablation catheter 100 includes a balloon arrangement 101 which includes a balloon section 115. The balloon section 115, when inflated, forces a transducer 120 against the aorta 20 at the aortorenal junction. The balloon arrangement 101 may include a tapered and pliant proximal member 117 that serves to enhance positional stability of the transducer 120 during and ablation procedure. The balloon arrangement 101 and transducer 120 are supported at a distal end of a catheter shaft 104. A lumen arrangement of the shaft 104 typically includes a conductor arrangement electrically coupled to the transducer 120 and an inflation lumen fluidly coupled to the balloon section 115.

Rather than using an artificial reflector, the embodiment illustrated in FIG. 8 employs an organ of the body to serve as a reflector. According to the representative embodiment shown in FIG. 8, the patient's kidney 10 and/or the main bifurcation serves as a reflector of acoustic energy transmitted by the transducer 120 positioned at the ostium 117 of the renal artery 12. Because the separation distance between the ostium 117 of the renal artery 12 and the kidney 10/main bifurcation varies among patients, a variable frequency acoustic transducer 120 is preferably used to provide for adjustment of the acoustic energy wavelength to achieve resonance.

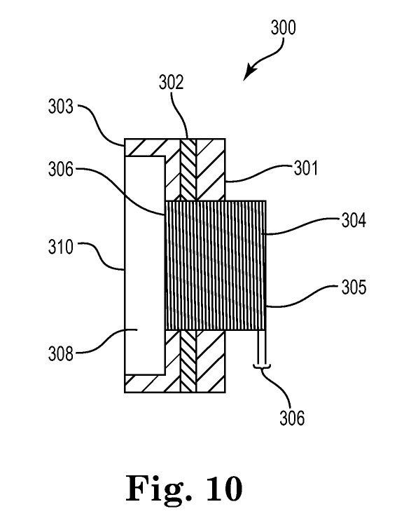

FIG. 9 shows an embodiment of the transducer 120 shown in FIG. 8. In the embodiment of FIG. 9, the transducer 120 is positioned between the balloon section 115 and the pliant proximal member 117. The transducer 120, according to some embodiments, includes a multiplicity of acoustic generator elements 120′ distributed circumferentially about a central axis, c, of the transducer assembly. The acoustic generator elements 120′ may be configured as phased array acoustic transducers comprising a multiplicity of individual acoustic wave generator elements supported by a flexible circuit substrate and arranged in a spaced apart relationship about the circumference of the transducer assembly.

The acoustic generator elements 120′ are oriented so that each produces longitudinally oriented acoustic waves that travel along an outer wall of the renal artery 12 and impinge on the main bifurcation and/or kidney 10. In some embodiments, each of the acoustic generator elements 120′ comprises a variable frequency acoustic generator element, which allows for varying the wavelength of emitted acoustic energy for each acoustic generator element 120′ according to the separation, L, between each element 120′ and portion of the main bifurcation and/or kidney 10.

In the following example, it is assumed that the separation distance between the ostium 117 and main bifurcation proximate the kidney 10 for a given patient is 3.8 cm and the speed of sound in tissue is 1500 m/s. Based on these assumptions, the resonant frequencies needed for the first two modes (R₁ and R₂) when the separation, L, is 3.8 cm are 9.868 kHz and 29.605 kHz, respectively. The resonant frequencies needed for the third and fourth modes (R₃ and R₄ not shown) are 49.342 kHz and 69.078 kHz, respectively. As discussed previously, an electromagnetic vibrator, such as that shown in FIG. 10, is preferably used for relatively low frequency ablation applications. It is understood that the length of the renal artery 12 can vary significantly among patients, but that most patients have at least one main renal artery 12 large enough to accommodate one or more components of an ablation catheter according to embodiments of the disclosure.

FIG. 10 illustrates an electromagnetic vibrator 300 in accordance with various embodiments. The electromagnetic vibrator 300 shown in FIG. 10 includes a housing 302 formed from an electrically insulating material. A rear section 301 of the housing 302 is configured to support a coil 304, which may include a magnetic core in some embodiments and exclude a magnetic core in others. Coil leads 306 are electrically coupled to the coil 304 typically at a rear section 305 of the coil 304. A front section 303 of the housing 302 includes a recess 308 that extends to a front surface 306 of the coil 304. A thin metal membrane 310 extends across the recess 308 of the front section 303 of the housing 302.

For operation below about 100 kHz, the coil 304 can include a magnetic core, and the membrane 310 may be formed of a magnetic material. In this case, the membrane 310 is pulled into a vacuum space 308 by the coil 304. For operation above about 100 kHz, the coil 304 typically has no magnetic core, and the membrane 310 may be formed of a non-magnetic, electrically conductive metal. The membrane 310 is pushed away from the coil 306 due to the repulsion from eddy currents induced in the membrane 310. The coil 304 is preferably energized with sine wave current to launch acoustic compression waves in a desired direction, such as along an outer wall of the renal artery 12 in a manner previously described. The electromagnetic vibrator 300 may be configured as an EMAT (electromagnetic acoustic transducer) which, in general terms, is a transducer configured for non-contact acoustic wave generation and reception using electromagnetic mechanisms. An advantage of an electromagnetic acoustic transducer is that a couplant is not needed since the acoustic waves are directly generated within the transducer.

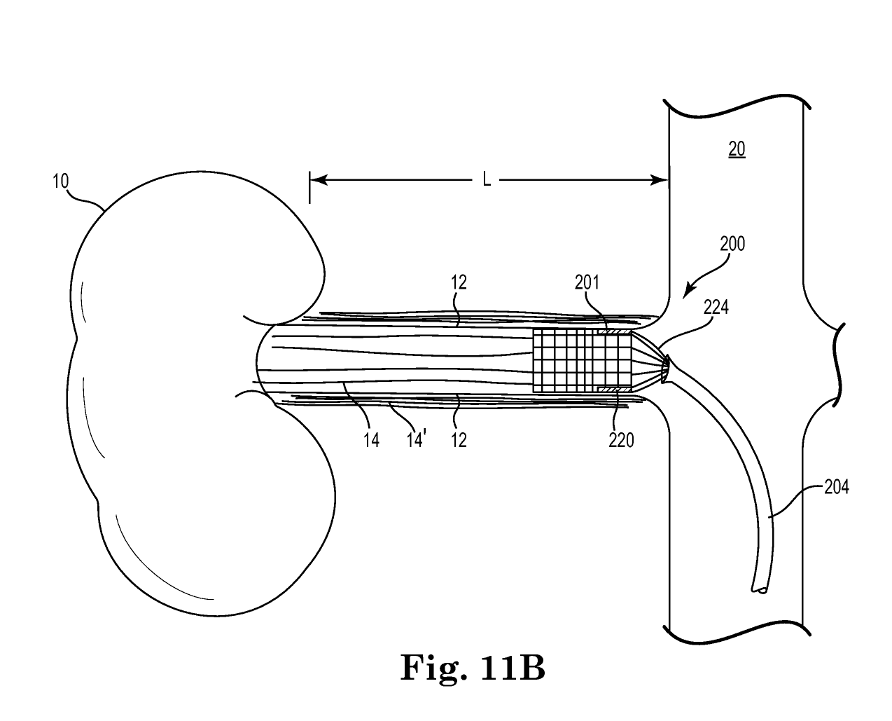

FIGS. 11A-11C illustrate an embodiment of a transducer assembly supported by a mesh structure in three different configurations in accordance with various embodiments. In the embodiment shown in FIGS. 11A-11C, an ablation catheter 200 includes a flexible shaft 204 having a lumen dimensioned to receive a transducer 220 supported by a cylindrical mesh structure 201 that is transformable between expanded and collapsed configurations. One or more conductors 224 are coupled to the transducer 220 and extend within a lumen of the shaft 204 to a proximal end of the ablation catheter 200. Preferably, the transducer 220 includes a multiplicity of individual spaced-apart transducers 220′ (see FIG. 11) distributed about a periphery of the cylindrical mesh structure 201. Each of the individual transducers 201′ is preferably coupled to one of the conductors 224.

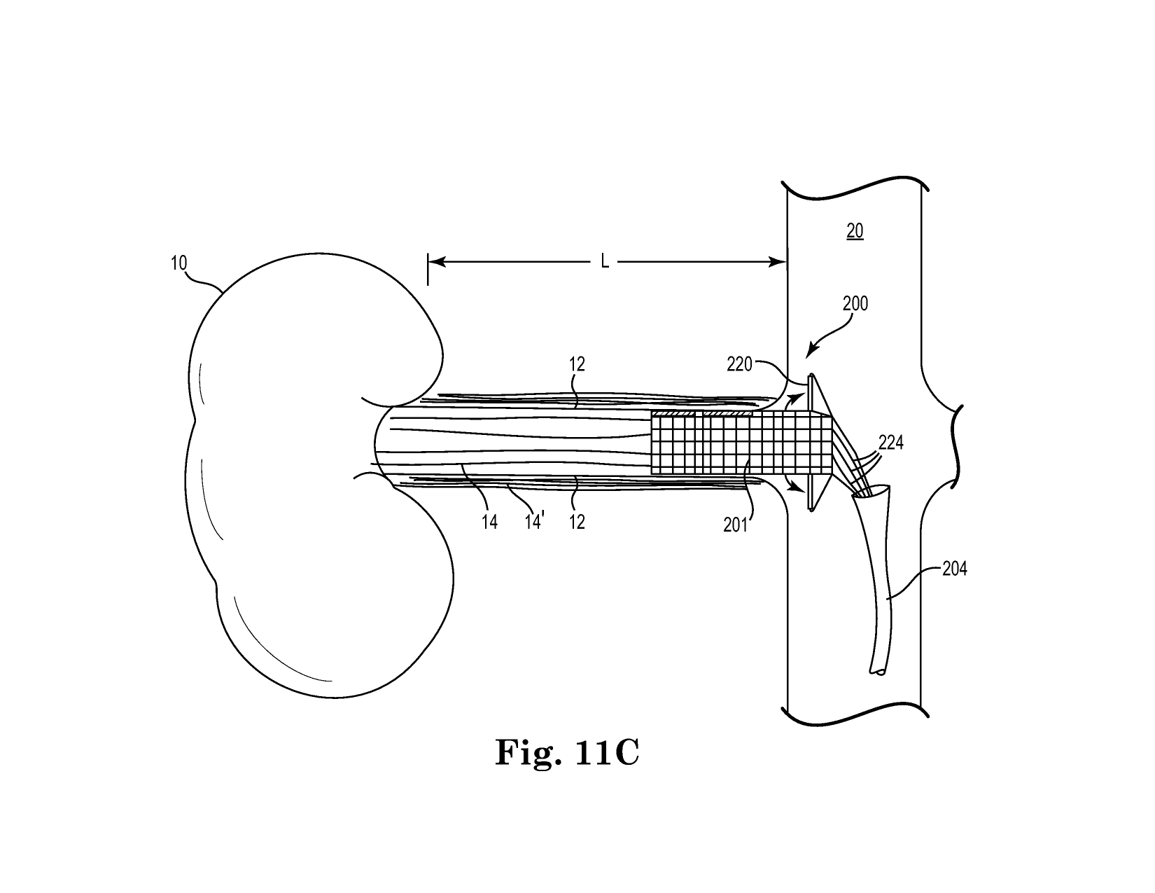

As is best seen in FIG. 11C, the transducer 220 is preferably mounted to a folding mechanism that transforms the transducer 220 from a low-profile introduction configuration to an expanded deployed configuration. When in the low-profile introduction configuration, such as shown in FIG. 11B, the transducer 220 lies approximately flush with the external surface of the mesh structure 201. When in the expanded deployed configuration, such as shown in FIG. 11C and FIG. 12, the transducer 220 unfolds outwardly.

The cylindrical mesh structure 201 provides the requisite structural integrity to support the transducer 220 yet is transformable between a low-profile introduction configuration and an expanded deployed configuration. The mesh structure 201 allows for perfusion of blood flow through the renal artery 12 during the ablation procedure. In some embodiments, the mesh structure 201 is configured as a self-expanding structure constructed of a suitable material, such as a nitinol alloy, a spring-like metal or alloy, or superelastic memory material, for example. In other embodiments, the mesh structure 201 need not be configured as a self-expanding structure, but is expandable and collapsible in response to manual manipulation of an actuator, such as a push/pull member.

FIG. 11A shows the distal end of the catheter's shaft 204 being advanced into the renal artery 12 to a position biased more toward the ostium 115 than the kidney 10. FIG. 11B shows deployment of the mesh support structure 201 within the renal artery 12. One approach to deploying the mesh structure 201 in the renal artery 12 involves advancing the distal end of the mesh structure 201 out of the distal end of the shaft 204 and allowing a proximal portion of the mesh structure 201 to expand into engagement with the wall of the renal artery 12 while retracting the shaft 204 into the aorta 20. With the mesh structure 201 removed from the shaft 204, the distal section of the expandable mesh structure 201 engages the renal artery wall, preferably with a modest bias force to enhance stability of the transducer 220 during the ablation procedure.

The expandable mesh structure 201 is preferably positioned in the renal artery 12 such that a proximal section of the expandable mesh structure 201 is positioned outside of the renal artery 12, extending partially into the aorta 20. This positioning of the expandable mesh structure 201 allows the transducer 220 transform from its low-profile introduction configuration to its expanded deployed configuration. A hinge mechanism is preferably used to hingedly connect the transducer 220 the proximal end of the expandable mesh structure 201. The transducer 220 is preferably dimensioned so that its effective diameter (i.e., transducer height plus mesh structure height in the vertical plane) is greater than that of the renal artery 12, which allows acoustic energy transmitted by the transducer 220 to propagate along an outer wall of the renal artery 12 and within the perivascular renal nerves tissue 14′ adjacent thereto.

FIG. 12 shows the transducer 220 illustrated in FIGS. 11A-11B in its deployed configuration. The transducer 220 shown in FIG. 12 comprises a multiplicity of spaced-apart transducer elements 201′ distributed circumferentially about the periphery of the expandable mesh structure 201. Each of the transducer elements 201′ is supported by a tab 201′, which may be formed of the same material as the mesh structure 201 or other self-expanding material. Each of the transducer elements 201′ is preferably a variable frequency transducer coupled to an individual conductor, allowing the wavelength of acoustic energy transmitted by each transducer element 201′ to be adjusted to achieve resonance. After completing the ablation procedure, the expandable mesh structure 201 is drawn into the distal end of the shaft 204, which may have a funnel shaped to facilitate collapsing of the expandable mesh structure 201 during retraction into the lumen of the shaft 104. The ablation catheter 200 may then the advanced into the patient's contralateral renal artery 12 to ablate perivascular renal nerve tissue adjacent to the contralateral renal artery 12. After denervating both renal arteries 12, the ablation catheter 200 is removed from the patient's body.

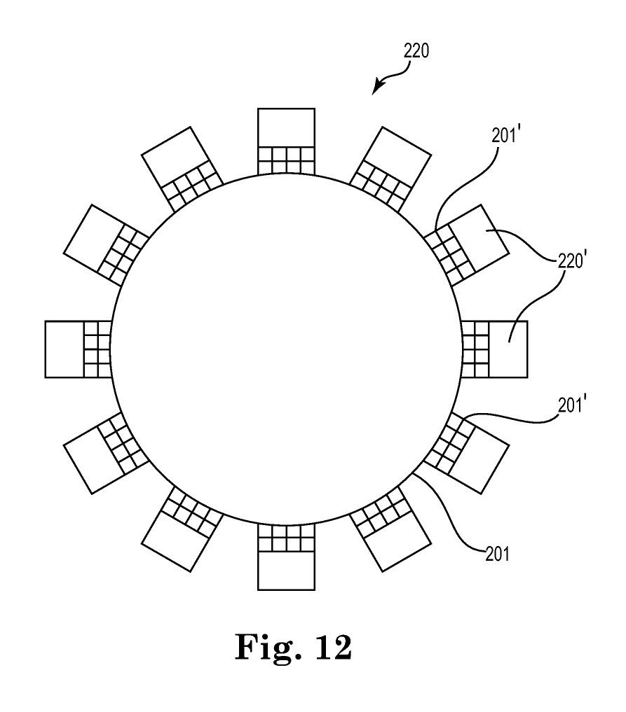

FIG. 13 illustrates an ablation catheter 350 which includes a cylindrical ultrasound transducer 352 and a positioning arrangement 354 provided at a distal end of a flexible shaft 356 of the ablation catheter 350 in accordance with various embodiments. The ultrasound transducer 352 may be implemented as a thin wall cylindrical transducer or a rotating flat transducer that is fitted into the distal end of the ablation catheter 200. The ablation catheter 350 may include features and functionality of the ultrasound ablation and/or imaging catheters disclosed in commonly owned co-pending U.S. patent application Ser. No. 13/086,116 filed Apr. 13, 2011, which is incorporated herein by reference.

In the embodiment shown in FIG. 13, the ultrasound transducer 352 is positioned within a lumen of the renal artery 12 and operates in a manner different from the previously described transducers for inducing resonance in renal nerves. Instead of generating relatively low frequency acoustic energy in the kilohertz range, the ultrasound transducer 352 generates high frequency ultrasonic energy in the megahertz range (e.g., 10 MHz). The ultrasound transducer 352 is preferably centered within the renal artery 12 by the positioning arrangement 354, which may be a balloon, mesh structure, or other expandable structure. The ultrasound transducer 352 is configured to emit high frequency acoustic energy radially outward from the periphery of the transducer 352, penetrating the wall of the renal artery 12 and the perivascular renal nerve tissue 14′ adjacent thereto. The acoustic energy emitted by the ultrasound transducer 352 excites the renal nerves 14 running substantially parallel to the wall of the renal artery 12 at resonance. In particular, the acoustic energy emitted by the ultrasound transducer 352 produces transverse waves on the renal nerves 14 at a resonance frequency of the renal nerves 14. One or more loops of high amplitude acoustic energy are created along a length of the renal nerves 14 that have an intensity sufficient to mechanically ablate the renal nerves 14.

The mechanical ablation mechanism implicated in the embodiment shown in FIG. 13 involves pressure from the high frequency ultrasound wave that pushes the renal nerve 12 radially outward away from the wall of the renal artery 12. The outward pressure creates a constriction or node. A loop of high amplitude acoustic energy can be created at a reflector, such as a kidney or bifurcation. A second node can be created using a second ultrasound transducer 352 spaced at a known length along the catheter's shaft 356. In this representative scenario, an acoustic wave having a frequency that is an integral number of half wavelengths will generate one or more loops or intensity maxima between the two nodes created by the two ultrasound transducers 352. In this embodiment, the nerve resonant frequency is generated at the rate of pulses (on and off bursts of ultrasound energy) that interrupt the outward pressure between bursts, causing the renal nerve 12 to move radially in and out. The burst rate is changed to find and achieve resonance. The bursts can be generated at one of the nodes (or both if they are phased properly). Although the speed of the transverse waves on the renal nerves 12 is typically unknown, resonance can be found by scanning the burst rate.

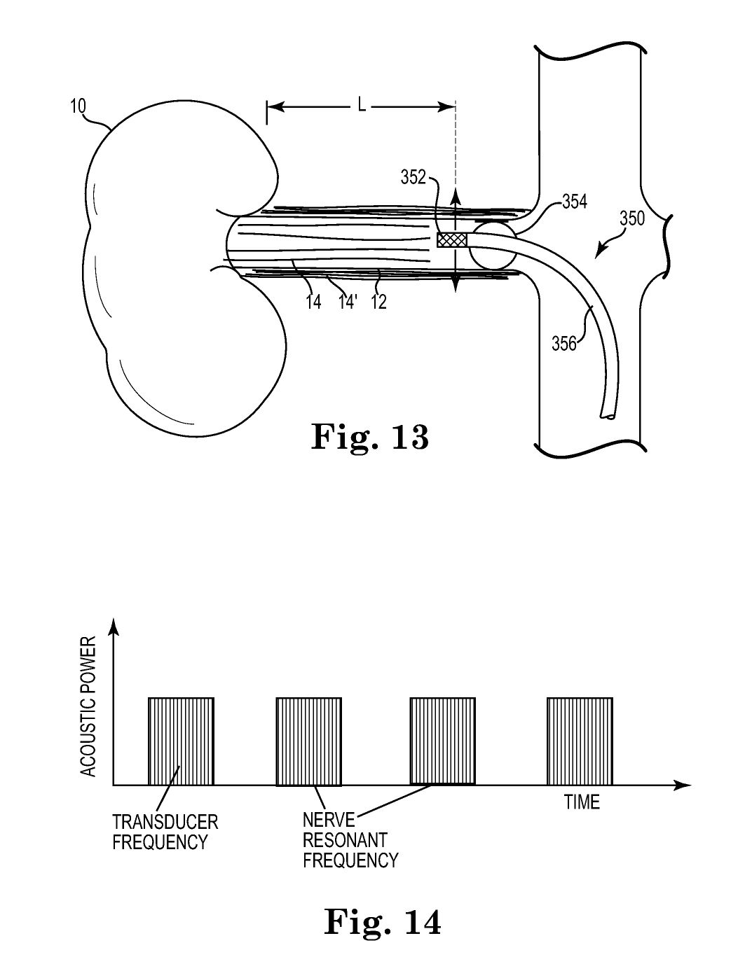

As is shown in FIG. 14, the ultrasound transducer 352 generates bursts of ultrasound energy at the resonance frequency of the transducer (e.g., 10 MHz). The resonant frequency of the transducer 352 is chosen to penetrate tissue to at least the depth of the renal nerves. These ultrasound energy bursts at the resonance frequency of the transducer are repeated at the expected resonance frequency of the transverse waves on the renal nerves 14. The pulse repetition rate (pulse frequency) may be varied in order to find the resonance frequency of the renal nerves 14. The acoustic pulse creates an outward pressure on the renal nerves 14 that is gated on and off to generate the transverse wave on the renal nerves 14. Acoustic pulses are repeated at the resonant frequency of the renal nerves 14 to generate a standing wave on the nerves 14. The kidney 10 and/or main bifurcation serve as a reflector which produces the standing wave on the nerves 14. A parameter such as reflected power at the transducer frequency may be monitored to detect renal nerve resonance.

As previously discussed, and in accordance with various embodiments, the distal end of the ablation catheter 350 shown in FIG. 13 can incorporate a multiplicity of cylindrical ultrasound transducers 352. Each of the spaced apart transducers 352 can be operated to create a node on the renal nerves 12, and a standing wave can be generated between the two nodes without the need for a reflector (e.g., the kidney 10 and/or main bifurcation).

According to other embodiments, an ultrasound ablation catheter may incorporate a wire that is coupled to a high-frequency vibration generator and vibrated longitudinally to impact against a metal frame at the distal end of an ablation catheter. This apparatus generates small amplitude, high frequency vibrations that succeed in ablating tissues proximate the distal end of the catheter. In some embodiments, a micro-motor can be incorporated at the distal end of an ablation catheter and driven to vibrate a wire or other structure at the distal end of the ablation catheter. In embodiments that use an external vibrator or a micro-motor, it is desirable that these excitation sources include a frequency tuning capability.

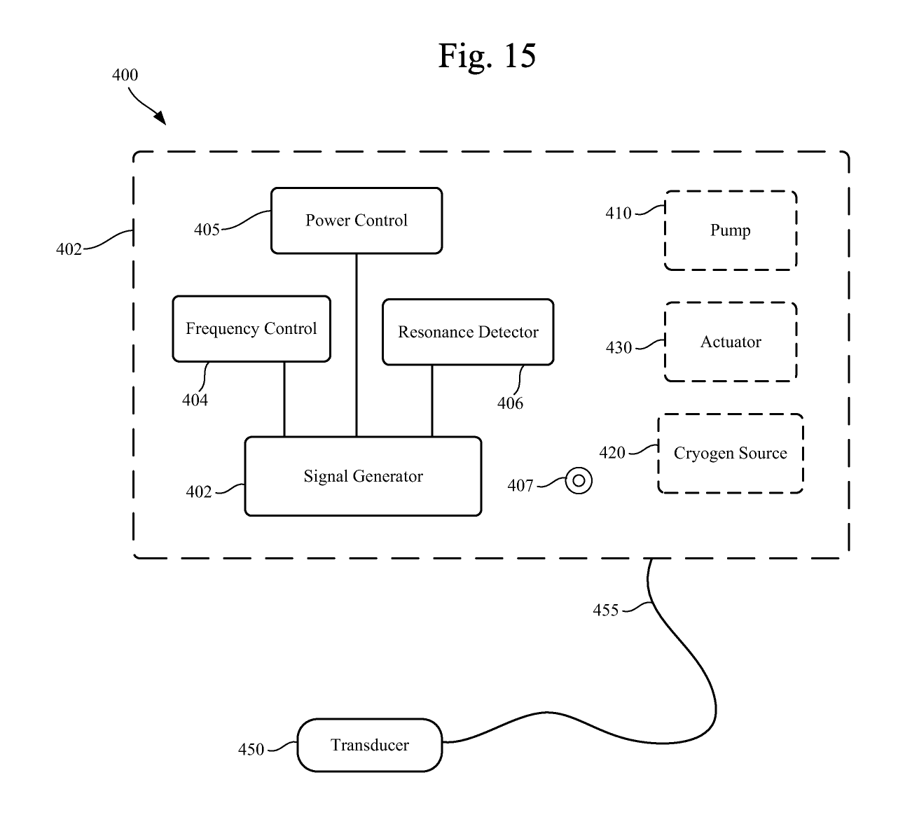

Referring now to FIG. 15, there is illustrated a system 400 for ablating tissues of the body, such as renal nerve tissue, using vibratory action resulting from acoustic wave excitation of the target tissue in accordance with various embodiments. The system 400 shown in FIG. 15 includes a number of external components and internal components. An external system 402 includes a signal generator 402 coupled to a frequency control 404, a power control 405, and a resonance detector 406. The signal generator 402 preferably includes an oscillator configured to generate an electrical signal that is transmitted to a transducer 450 provided at a distal end of an ablation catheter 455.

In some embodiments, the signal generator 402 and the transducer 450 cooperate to produce acoustic energy having a specified wavelength based on a predetermined distance separating the transducer 450 and a reflector, such as an artificial or biological reflector as described herein. This predetermined distance is equal to an odd number of quarter wavelengths of the acoustic energy emitted by the transducer 450. The acoustic energy emitted by the transducer 450 builds up to resonance at a point of reflection defined by a location of the reflector, such that the amount of energy buildup is sufficient to mechanically ablate target tissue in the vicinity of the reflector.

The frequency control 404 may be adjusted to adjust the wavelength of the acoustic energy emitted by the transducer 450. During the wavelength adjustment procedure, the power of the emitted acoustic energy is preferably relatively low, as selected by the power control 405. The resonance detector 406 can be used to detect resonance, such as by monitoring a parameter such as reflected power at the transducer resonant frequency. When resonance is detected, the power of the emitted acoustic energy is increased using the power control 405, preferably to a magnitude sufficient to mechanically ablate the target tissue.

In other embodiments, the signal generator 402 is configured to generate bursts of ultrasound energy at the resonance frequency of the transducer 450, which is chosen to penetrate the target tissue to a prescribed depth. The ultrasound energy bursts generated at the transducer resonance frequency are repeated at an expected resonance frequency of acoustic waves in or on the target tissue, such as transverse waves on the renal nerves. The frequency control 404 in this embodiment allows for adjustment of the pulse frequency, which may be varied in order to find resonance frequency of the target tissue. When the resonant frequency of the target tissue is determined, such as by use of resonance detector 406, acoustic pulses are repeated at the resonant frequency to generate a standing wave in the target tissue. A power control 405 of the signal generator 402 is adjusted to increase the amplitude of the standing wave so that disruptive vibration of the target tissue results in ablation of the target tissue.

In embodiments that employ a balloon reflector, such as that shown in FIG. 7, the system 402 typically includes a pump 410 which is fluidly coupled to an inflation lumen of the catheter shaft 455. The pump 410 is controlled to inflate and deflate the balloon reflector as described in the context of the embodiment shown in FIG. 7. A cryogen source 420 may be included in the system 402 for embodiments that employ a balloon reflector whose acoustic reflection characteristics can be enhanced by formation of ice at the reflector location. The system 402 may further include an actuator 430 in embodiments in which an expandable mesh structure or other expandable structure that supports an acoustic transducer is transformed between low-profile introduction configuration and an expanded deployed configuration. In such embodiments, such as those shown in FIGS. 11A-12, a push/pull elongated member extending through the catheter shaft 455 may be manipulated by a clinician to facilitate expansion and collapsing of the expandable mesh structure.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vasculature (e.g., cardiac and urinary vasculature and vessels), and other tissues of the body, including various organs. Various embodiments disclosed herein are generally described in the context of mechanical ablation using an electrical energy supplied to an acoustic vibrator or generator or to an ultrasound transducer. Other energy sources can be pulsed to generate standing waves on the renal nerves or in target tissue that supports standing waves, such as optical, electrical, thermal, and mechanical sources.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a flexible shaft comprising a proximal end, a distal end, a length, and a lumen arrangement extending between the proximal and distal ends, the length of the shaft sufficient to access a renal artery relative to a percutaneous access location of the body;
a positioning structure provided at a distal end of the shaft; and
a transducer supported by the positioning structure and arranged to emit acoustic energy so that it propagates axially along an outer surface of the renal artery to impinge a reflector, the acoustic energy emitted by the transducer producing standing waves on perivascular renal nerves and one or more loops of high amplitude acoustic energy sufficient to ablate the perivascular renal nerves.

2. The apparatus of claim 1, wherein a first point of reflection is created by the reflector situated at a predetermined distance from the transducer equal to an odd number of quarter wavelengths of the acoustic energy emitted by the transducer.

3. The apparatus of claim 1, wherein the transducer is configured to emit a circular beam of acoustic energy along the outer surface of the renal artery that impinges on a circumferential surface of the reflector.

4. The apparatus of claim 1, wherein:
the transducer comprises a variable frequency transducer; and
the frequency of the transducer is adjustable within a range of frequencies that achieve resonance of the renal nerves.

5. The apparatus of claim 1, wherein:
the transducer comprises a plurality of variable frequency transducer elements positionable about a circumferential region of the renal artery;
a separation distance between the reflector and at least some of the transducer elements differs; and
the frequency of acoustic energy emitted by each of the transducer elements can be tuned to a resonance frequency based on the separation distance between the reflector and each of the transducer elements.

6. The apparatus of claim 1, wherein the reflector comprises an organ of the body.

7. The apparatus of claim 1, wherein the reflector is a component of the catheter.

8. The apparatus of claim 1, wherein the transducer comprises an electromagnetic vibrator.

9. The apparatus of claim 1, wherein at least a portion of the positioning structure is configured to abut an ostium of the renal artery.

10. The apparatus of claim 1, wherein the positioning structure comprises a mesh structure configured to self-expand from a low-profile introduction configuration to a deployed configuration when actuated within the renal artery.

11. The apparatus of claim 1, wherein:
the positioning structure comprises a balloon apparatus fluidly coupled to the lumen arrangement, the balloon apparatus comprising:
a first balloon section dimensioned for abutting engagement with a wall of the aorta at an ostium of the renal artery; and
a second balloon section dimensioned for deployment within the renal artery and comprising a bulge feature at the distal end of the second balloon section, the bulge feature situated at the predetermined distance from the transducer when the second balloon section is pressurized, the bulge feature dimensioned to expand to a radius greater than a radius of the renal artery and cause formation of a bump in the renal artery when the second balloon section is pressurized, the bulge feature forming the renal artery bump which serves as the reflector; and
the transducer is positioned proximate the first balloon and configured for forced abutment relative to the wall of the aorta at the renal artery ostium in response to inflation of the first balloon;
wherein the acoustic energy emitted by the transducer builds up to resonance at the point of reflection defined by the renal artery bump, the amount of acoustic energy build up sufficient to ablate perivascular renal nerves in the vicinity of the renal artery bump.

12. The apparatus of claim 11, wherein the second balloon section is configured to receive a cryogen causing formation of ice thereon, the ice serving to enhance reflection of the emitted acoustic energy by the bulge feature.

13. An apparatus, comprising:
a flexible shaft comprising a proximal end, a distal end, a length, and a lumen arrangement extending between the proximal and distal ends, the length of the shaft sufficient to access target tissue of the body relative to a percutaneous access location, the target tissue capable of supporting standing waves;
a positioning structure provided at a distal end of the shaft; and
a transducer supported by the positioning structure and arranged to emit acoustic energy so that it impinges a reflector within or proximate the target tissue, the acoustic energy emitted by the transducer producing standing waves in the target tissue and one or more loops of high amplitude acoustic energy sufficient to ablate the target tissue.

14. The apparatus of claim 13, wherein a first point of reflection is created by the reflector situated at a predetermined distance from the transducer equal to an odd number of quarter wavelengths of the acoustic energy emitted by the transducer.

15. The apparatus of claim 13, wherein:
the transducer comprises a variable frequency transducer; and
the frequency of the transducer is adjustable within a range of frequencies that achieve resonance of the target tissue.

16. The apparatus of claim 13, wherein the reflector comprises an organ of the body.

17. The apparatus of claim 13, wherein the reflector is an artificial reflector.

18. A method, comprising:
positioning a transducer within or proximate target tissue that supports standing waves at a location relative to a reflector;
emitting acoustic energy by the transducer so that it impinges the reflector; and
ablating the target tissue by producing standing waves in the target tissue and one or more loops of high amplitude acoustic energy sufficient to ablate the target tissue.

19. The method of claim 18, comprising adjusting a frequency of the emitted acoustic energy to achieve resonance of the target tissue.

20. The method of claim 18, wherein:
the transducer is positioned within a renal artery;
the acoustic energy is emitted so that it propagates axially along an outer surface of the renal artery to impinge the reflector; and
perivascular renal nerves are ablated by producing standing waves on the renal nerves and one or more loops of high amplitude acoustic energy sufficient to ablate the renal nerves.

21. An apparatus, comprising:
a catheter comprising a flexible shaft having a proximal end, a distal end, a length, and a lumen arrangement extending between the proximal and distal ends, the length of the shaft sufficient to access a renal artery relative to a percutaneous access location of the body;
a cylindrical ultrasound transducer provided at a distal end of the shaft and dimensioned for placement within a lumen of the renal artery; and
a positioning structure provided at a distal end of the shaft and transformable between a low-profile introduction configuration and a deployed configuration, the positioning structure configured to center the transducer in the lumen of the renal artery when in the deployed configuration;
wherein the transducer is configured to generate bursts of ultrasound energy and repeatedly emit the ultrasound energy bursts at a resonance frequency of the renal nerves to generate standing waves on the renal nerves of sufficient amplitude to mechanically ablate the renal nerves.

22. The apparatus of claim 21, wherein the cylindrical ultrasound transducer is configured to emit acoustic energy to a biological reflector.

23. The apparatus of claim 21, comprising a plurality of the cylindrical ultrasound transducers spaced apart from one another at the distal end of the shaft, wherein the standing waves on the renal nerves are generated between nodes created by the plurality of transducers.

24. A method, comprising:
positioning a cylindrical ultrasound transducer in a lumen of a renal artery at a central location of the lumen;
generating bursts of ultrasound energy; and
repeatedly emitting the ultrasound energy bursts at a resonance frequency of the renal nerves to generate standing waves on the renal nerves of sufficient amplitude to mechanically ablate the renal nerves.
